(19) United States
(12) Patent Application Publication
(10) Pub. No.: US 2026/0294557 A1
(43) Pub. Date: Oct. 1, 2026

(54) BONE MOUNT END EFFECTOR

(71) Applicant: Mazor Robotics Ltd., Caesarea (IL)

(72) Inventors: Yohai Ben-Noun, Pardes Hanna-Karkur (IL); Gillan M. Grimberg, Ramat Gan (IL); Amir Keret, Atlit (IL); Jaffar Hleihil, Zichron Yaakov (IL); Nimrod Dori, Atlit (IL); Gal Barazani, Tirat Carmel (IL)

(21) Appl. No.: 19/489,138

(22) PCT Filed: Jun. 6, 2024
(86) PCT No.: PCT/IL2024/050560

§ 371 Date: Dec. 1, 2025

Related U.S. Application Data
(60) Provisional application No. 63/471,455, filed on Jun. 6, 2023.

(51) Int. Cl. A61B 34/30; A61B 17/00; A61B 17/56; A61B 90/00

(52) U.S. Cl. CPC A61B 34/30; A61B 17/56; A61B 90/06; A61B 2017/00022; A61B 2017/00119; A61B 2017/00477; A61B 2090/066

(57) ABSTRACT

A system according to at least one embodiment of the present disclosure includes a robotic arm, an end effector, a bone mount and a bone mount interface. The end effect has a proximal end and a distal end with the proximal end of the end effector being connected to the robotic arm. The bone mount is attachable to an anatomical element at one end of the bone mount and the bone mount interface is coupled to the distal end of the end effector via a proximal end of the bone mount interface and attached to another end of the bone mount via a distal end of the bone mount interface. The bone mount interface is configured to add a degree of freedom to the end effector.

### BACKGROUND

The present disclosure is generally directed to surgical systems, and relates more particularly to robotic surgical devices.

Surgical robots may assist a surgeon or other medical provider in carrying out a surgical procedure, or may complete one or more surgical procedures autonomously. Providing a rigid connection between an end effector of the surgical robot and a bone mount placed on a patient allows for the most accurate registration of the patient to the surgical robot.

### BRIEF SUMMARY

Example aspects of the present disclosure include:

A system according to at least one embodiment of the present disclosure comprises a robotic arm, an end effector, a bone mount and a bone mount interface. The end effect has a proximal end and a distal end with the proximal end of the end effector being connected to the robotic arm. The bone mount is attachable to an anatomical element at one end of the bone mount and the bone mount interface is coupled to the distal end of the end effector via a proximal end of the bone mount interface and attached to another end of the bone mount via a distal end of the bone mount interface. The bone mount interface is configured to add a degree of freedom to the end effector.

Any of the aspects herein, wherein the anatomical element includes one or more vertebrae.

Any of the aspects herein, further comprising one or more sensors disposed at the distal end of the end effector.

Any of the aspects herein, wherein the one or more sensors include a force/torque sensor disposed at the distal end of the end effector configured to output a force signal in accordance with a force exerted on the bone mount.

Any of the aspects herein, further comprising control circuitry configured to receive the force signal and output a control signal based on the force exerted on the bone mount.

Any of the aspects herein, wherein the control signal includes activating an alarm when the force signal exceeds a predetermined threshold.

Any of the aspects herein, wherein the control signal includes releasing the bone mount from the bone mount interface when the force signal exceeds a predetermined threshold.

Any of the aspects herein, wherein the one or more sensors provide feedback to the robotic arm, allowing the robotic arm to adjust its position and orientation based on movement of the anatomical element.

Any of the aspects herein, wherein the bone mount includes a locking mechanism. configured to be selectively and repeatedly coupled to and uncoupled from the bone mount interface.

Any of the aspects herein, wherein the bone mount includes a ball and socket mechanism.

Any of the aspects herein, wherein the degree of freedom is a rotational degree of freedom.

Any of the aspects herein, wherein the degree of freedom is a translational degree of freedom.

Any of the aspects herein, wherein the bone mount interface is mechanically coupled to the distal end of the end effector to provide a rigid connection between the end effector and the bone mount.

Any of the aspects herein, wherein the bone mount includes a kinetic interface or a clamping interface.

Any of the aspects herein, further comprising an arm guide provided between a proximal end and a distal end of the robotic arm and adjacent to the end effector, wherein the arm guide is configured to accommodate a surgical tool.

A system according to at least one embodiment of the present disclosure comprises one or more processors, at least one robotic arm and a memory storing data for processing by the one or more processors that, when processed by the one or more processors, causes the one or more processors to determine, based on first sensor data, a first force exerted by a bone mount interface onto a bone mount attached to an anatomical element, determine, based on second sensor data, a second force different from the first force, exerted by the bone mount interface onto the bone mount and output a control signal based on the second force exerted on the bone mount. The bone mount interface is coupled to an end effector of the at least one robotic arm.

Any of the aspects herein, wherein the control signal includes activating an alarm when the second force exceeds a predetermined threshold.

Any of the aspects herein, wherein the control signal includes releasing the bone mount from the bone mount interface when the second force exceeds a predetermined threshold.

A method according to at least one embodiment of the present disclosure comprises attaching one end of a bone mount to an anatomical element, coupling a bone mount interface to a distal end of an end effector of a robotic arm via a proximal end of the bone mount interface and attaching another end of the bone mount to a distal end of the bone mount interface. The bone mount interface is configured to add a degree of freedom to the end effector.

Any of the aspects herein, further providing one or more sensors at the distal end of the end effector.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a block diagram of aspects of the system according to at least one embodiment of the present disclosure;

FIG. 3 is a perspective diagram of a robotic arm with a bone mount end effector according to at least one embodiment of the present disclosure;

FIG. 4 is a detail perspective view of a portion of the robotic arm with the bone mount end effector according to at least one embodiment of the present disclosure;

FIG. 5 is a detail perspective view of a portion of the robotic arm with the end effector including the bone mount interface according to at least one embodiment of the present disclosure;

FIG. 6 is a flowchart of a method for operating the robotic arm with the bone mount end effector according to at least one embodiment of the present disclosure; and

FIG. 7 is a flowchart of a method for measuring a location of an end effector of a robotic arm relative to a bone mount and navigating the robotic arm relative to a patient anatomy using the bone mount according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

During surgical procedures such as spine fixation surgery, for example, if a robotic arm is attached rigidly to a patient's bone (e.g., a vertebra) either directly to the bone, or on a structure attached rigidly to the bone, and a significant force is exerted by the robotic arm or the surgeon onto a surgical tool, there is a danger that the force to the surgical tool on the bone may be sufficient in some cases, to detach a bone-mounting element from the bone such that the robotic arm's position is no longer defined relative to the bone. Even if complete detachment does not occur or even if no movement of the bone-mounting element occurs, a loss of the defined spatial relationship between the robotic arm and the bone still may exist. Issues with the above may be addressed with embodiments of the present disclosure presented herein.

According to embodiments of the present disclosure, a robotic arm with a bone mount end effector is a device that may be used in surgical procedures to provide a greater precision and control during the surgical procedures involving the manipulation of bone tissue, for example. The robotic arm is typically mounted on the operating table and is usually controlled by a computer of a handheld device operated by a surgeon. The robotic arm can be programmed to move in specific patterns or follow a predetermined path, allowing the surgeon to perform complex surgical procedures with a high degree of accuracy.

The bone mount end effector includes a bone mount interface and a bone mount. The bone mount is an attachment that can be affixed to the patient's anatomy (e.g., a bone) allowing the robotic arm to precisely position surgical tools, for example, during a surgical procedure. The bone mount is designed to hold the bone in a specific position and orientation, allowing the surgeon to make accurate incisions based on pre-operative planning. According to embodiments of the present disclosure, the bone mount is designed to securely attach to the bone tissue, without causing any damage or undue stress. Moreover, the bone mount is designed to interface with the robotic arm, allowing the surgeon to manipulate and control the robotic arm with precision and accuracy.

The bone mount interface on the other hand, is a mechanism that acts as a connection point between the bone mount and the robotic arm to provide a secure and stable connection between the robotic arm and the patient's bone. The bone mount interface is designated to provide a stable and secure attachment while still allowing for a wide range of motion and flexibility. According to embodiments of the present disclosure, the bone mount end effector can include various locking mechanisms between the bone mount interface and the bone mount for easy connection and reconnection. As such, the locking mechanisms are more flexible than conventional locking mechanisms. Moreover, the locking mechanism may include a clamp, a screw, a ball and socket, a kinematic interface, etc. to ensure that the bone mount remains in place during the surgical procedure. Moreover, the locking mechanism can be a manual locking mechanism or an electronic locking mechanism. Furthermore, the locking mechanism is easy to use and does not place excess force on the patient anatomy. Also, the locking mechanism connects easily and without forcing movement. According to an alternative embodiment of the present disclosure, a force or torque sensor is included that is configured to detect any skiving relative to the patient anatomy.

According to embodiments of the present disclosure, the distal end of the robotic arm to which the bone mount interface is attached, may include various sensors and feedback mechanisms to ensure accurate positioning and alignment during the surgical procedures by determining the location of the bone mount relative to the robotic arm. According to embodiments of the present disclosure, the bone mount interface provides the most accurate position of the patient, using feedback from the sensors to the robotic system. According to embodiments of the present disclosure, the location of where the bone mount is attached to the patient anatomy can be monitored with the sensors for maximum accuracy. Thus, the sensors provide feedback to the robotic arm, allowing the robotic arm to adjust its position and orientation based on movement of the patient, the breathing of the patient, the movement of the patient bone, etc.

Moreover, the feedback mechanism is provided such that the bone mount interface is released from the bone mount if a threshold level of force or distance is received by a processor of the robotic system. Also, the feedback mechanism may be provided such that an alarm is caused to be generated by the processor of the robotic system if a threshold level of force exerted by the surgeon or the robotic arm or a threshold distance is displaced by the bone mount is received by the processor.

By using a robotic arm with the bone mount end effector, surgeons can perform delicate and complex procedures with a greater degree of accuracy, reducing the risk of complications and improving patient outcomes. The bone mount interface is compatible with a variety of different bone mount designs in order to accommodate different surgical procedures and patient needs.

A robotic arm with the bone mount end effector has numerous advantages over traditional surgical methods. The robotic arm with the bone mount end effector can provide greater precision and accuracy, reduce the risk of human error and allow for less invasive procedures that result in faster recovery times and fewer complications.

According to embodiments of the present disclosure, mounting the robotic arm with reference to a patient's anatomy allows the robotic systems to have a better accuracy than robotic systems that are not patient mounted. When enabling a bone mount to a patient anatomy at the end effector of the robotic arm, a higher degree of accuracy can be achieved. This higher degree of accuracy is achieved by the relative geometry of the patient anatomy (e.g., vertebra) and the end effector being locked in place.

In accordance with embodiments of the present disclosure, the bone mount interface enables rigid connection between the end effector and the bone mount with minimum length. Moreover, the bone mount interface enables a simple connection with the patient since the bone mount interface can be used with a variety of bone mounts. Furthermore, the bone mount interface, when attached to standard robotic arms, adds an additional degree of freedom (DOF) to the standard robotic arms with little to no reconfiguration of the standard robotic arms. According to one embodiment of the present disclosure, the additional DOF is an added rotational DOF at the end effector after the arm guide of the robotic arm. In this case the arm guide remains stationary. According to an alternative embodiment of the present disclosure, the additional DOF is an added cartesian DOF at the end effector after the arm guide. In this case also, the arm guide remains stationary.

In one embodiment of the present disclosure, the bone mount includes orientation capabilities. Therefore, a registration process can register the location of the bone mount. In the registration process, the orientation of the vertebra to the bone mount is determined. This determination is made in several ways. After the registration process, the location of the bone mount interface relative to the bone mount is measured. Accordingly, a surgical procedure on one or two vertebrae above or below where the bone mount is located may be performed.

The registration of the bone mount is not the key feature of the present disclosure. For example, a reference frame to the bone mount is first registered. Afterwards, the navigation system such as an optical navigation system, is used to determine the location of the robotic arm. According to embodiments of the present disclosure, the incorporation of the bone mount interface enhances the features of the optically navigated robotic arm. Moreover, since the bone mount interface is used with the navigation system to determine location, various types of materials such as metal, plastic, etc. can be used for the bone mount.

Embodiments of the present disclosure provide technical solutions to one or more of the problems of (1) accurate mounting of a robotic arm to a patient anatomy and (2) inaccurate tracking of the robotic arm relative to the patient anatomy.

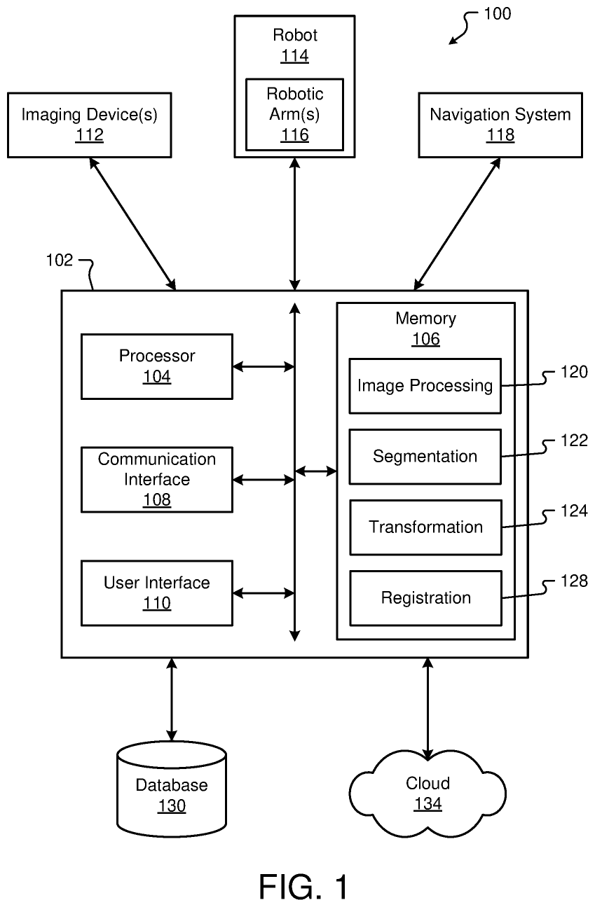

Turning first to FIG. 1, a block diagram of a system 100 according to at least one embodiment of the present disclosure is shown. The system 100 may be used to operate a robot 114 in order to provide a rigid connection between an end effector of the robot 114 and a bone mount placed on a patient to allow for the most accurate registration of the patient to the robot 114. In some examples, the system 100 may control, pose, and/or otherwise manipulate a surgical mount system, a surgical arm, and/or surgical tools attached thereto and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor XTM Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. In one example, the database 130 may comprise movement profiles for the robot 114 based on a select end effector that is attached to the robotic arm 116. These movement profiles may correspond to kinematic solutions for the robot 114 and/or defined positions of a surgical tool axis of select end effector relative to at least one of a surface of a tool block of the end effector and a rotation axis of a final joint/mount flange of the robotic arm 116. In some examples, the database 130 may store identifications of specific tool blocks and surgical tool axis orientations. In any event, the database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods (e.g., methods 600 and 700, etc.) described herein. The system 100 or similar systems may also be used for other purposes.

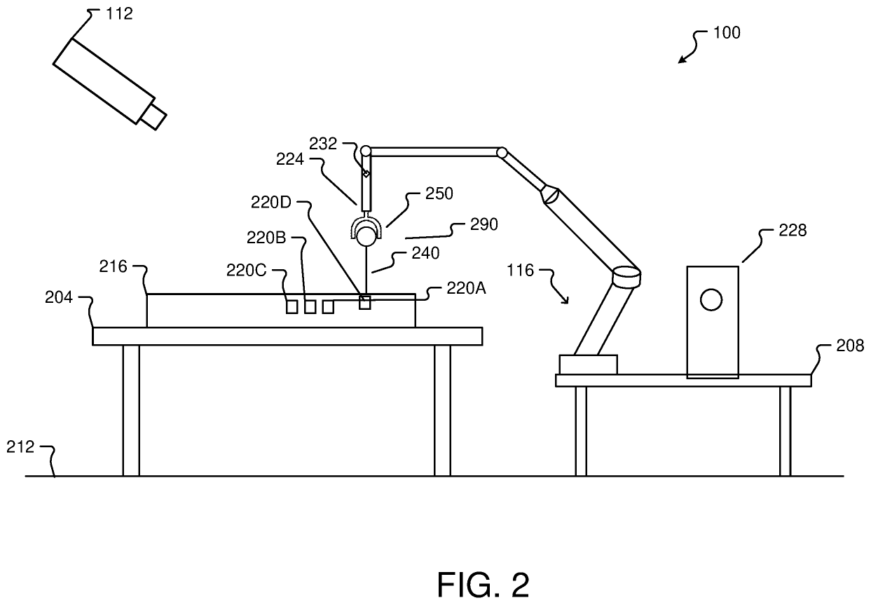

FIG. 2 is a block diagram of aspects of the system 100 according to at least one embodiment of the present disclosure. FIG. 2 illustrates a surgical environment, such as an operating room, including a patient table 204 and a robotic table 208. The patient table 204 and the robotic table 208 may be positioned on a floor 212 of the surgical environment. In some embodiments, the patient table 204 and/or the robotic table 208 may be mobile and capable of being moved around the surgical environment. For example, the robotic table 208 may be or comprise a cart that moves relative to the patient table 204, allowing the robotic table 208 (and the robotic arm 116) to be brought in or otherwise introduced to the surgical environment after preparations for a surgery or surgical task have been performed. The robotic table 208 may be brought into the surgical environment after preoperative imaging has been performed, for example, to help lessen congestion of the surgical environment. According to an alternative embodiment of the present disclosure, the robotic arm 116 may be attached to the patient table 204 itself and the robotic table 208 would not be need. Likewise, the robotic arm 116 could be free standing and include a base positioned on the floor 212.

A patient 216 may be positioned on the patient table 204. The patient 216 may have anatomical elements 220A-220D, which may be the subject of the surgery or surgical procedure. For example, the surgical procedure may be a spinal fusion, and the anatomical elements 220A-220D may be vertebrae of the spine. In some embodiments of the present disclosure, the patient 216 may be securely positioned on the patient table 204, such that the patient 216 and/or the anatomical elements 220A-220D cannot move relative to the patient table 204. While the discussion herein includes discussion of an anatomical element, it is to be understood that more or fewer anatomical elements may be present and may be identified and registered using methods discussed herein. Furthermore, it is to be understood that the methods and embodiments discussed herein may alternatively apply to a portion of an anatomical element (e.g., a spinous process of a vertebra).

The robotic table 208 includes the robotic arm 116 and an optical sensor 228. In some embodiments of the present disclosure, the robotic table 208 may include additional or alternative components. For example, the optical sensor 228 may not be positioned on the robotic table 208, and may instead be disposed in another location in the surgical environment (e.g., on the floor 212, mounted on a wall, positioned on another surgical table, etc.). The robotic table 208 may include additional surgical components such as surgical tools, and may include one or more cabinets, drawers, trays, or the like to house the surgical components. In some embodiments, the robotic table 208 may be mechanically decoupled or may be otherwise detached from the patient of the present disclosure table 204, such that the robotic table 208 and/or the surgical components thereon can move freely relative to the patient table 204, the patient 216, and/or the anatomical elements 220A-220C. In one embodiment, the robotic table 208 may be disposed a first distance from the patient table 204 (e.g., 0.5 meters (m), 1m, 1.5m, 2m, etc.).

The optical sensor 228 may be or comprise a sensor capable of detecting and/or tracking optics-based targets (e.g., illuminated objects, visual targets, etc.). In some embodiments, the optical sensor 228 may be or comprise a laser tracker. The laser tracker may project or emit a laser that may reflect off one or more targets and back toward the laser tracker. The reflected light may be received and processed by the computing device 102 and/or the navigation system 118 and may enable the computing device 102 and/or the navigation system 118 to determine the relative distance and/or pose of the target relative to the laser tracker based on, for example, the angle, intensity, frequency, and/or the like of the returning laser. In one embodiment, the laser tracker may include a tracking system that tracks the target as the target moves, such that the laser tracker can continuously aim a laser at the target and receive the reflected laser. The information of the reflected laser may be processed (e.g., by the computing device 102, by the navigation system 118, etc.) to identify and determine a change in pose of the target as the target moves relative to the optical sensor 228. Alternatively, the optical sensor 228 may be or comprise a 3D camera capable of identifying one or more 3D optical tracking targets. The 3D camera may be able to identify the 3D optical tracking targets based on a number of faces, designs, or patterns displayed by the 3D optical tracking targets. For example, the 3D camera may identify the optical tracking target based on different QR codes displayed on each surface of the 3D optical tracking target. The processor 104 may receive the identified faces, and may determine (e.g., using transformations 124) the pose of the optical tracking target within the surgical environment. For example, the identified faces may be compared to a predetermined (e.g., preoperative) pose of the surfaces, with the changes in pose of each faces used to determine the pose of the optical tracking target.

In some embodiments, the optical sensor 228 may be disposed proximate the robotic arm 116 (e.g., disposed 0.1m, 0.2m, 0.5m, 1m, 1.5m, 2m, etc. away from the robotic arm 116), such that the optical sensor 228 can view and track the robotic arm 116 in addition to any optical-based targets in the environment. Alternatively, the optical sensor 228 may be disposed within a portion of the robotic arm 116 (e.g., within the end effector 224). In such embodiments, the optical sensor 228 may be disposed in a predetermined configuration relative to the robotic arm 116, such that the pose of the robotic arm 116 may be determined based on internal readings generated by a sensor (e.g., using gyroscopes, accelerometers, etc.).

The robotic arm 116 may include an end effector 224. The end effector 224 may be or comprise a receptacle, mount, gripper, or other mechanical interface for interacting with a surgical tool or instrument. For example, the end effector 224 may interface with a surgical tool to manipulate a surgical tool (whether based on guidance from the navigation system 118 or not) to accomplish or to assist with a surgery or surgical procedure or task. In some embodiments of the present disclosure, the end effector 224 may include a bone mount interface 250. The bone mount interface 250 is a mechanism that acts as a connection point between a bone mount 240 and the robotic arm 116 to provide a secure and stable connection between the robotic arm 116 and an anatomical element 220. The bone mount interface 250 is designated to provide a stable and secure attachment while still allowing for a wide range of motion and flexibility.

In some embodiments of the present disclosure, the end effector 224 may include a tracking marker 232 disposed thereon. The positioning of the tracking marker 232 on the end effector 224 may enable the imaging devices 112 and/or the optical sensor 228 to track the pose of the end effector 224. For example, the tracking marker 232 may be a QR code and the computing device 102 and/or the navigation system 118 can use the identified QR code to determine a pose of the tracking marker 232. Based on the pose of the tracking marker 232 and a predetermined position of the tracking marker 232 on the end effector 224, the computing device 102 and/or the navigation system 118 may use the pose of the tracking marker 232 to determine a pose of the end effector 224 or, more generally, a pose of the robotic arm 116. According to a further embodiment of the present disclosure, the tracking marker 232 may be an optics-based target (e.g., illuminated objects, visual targets, etc.), whereby the optical sensor 228 may be or comprise a sensor capable of detecting and/or tracking the optics-based target.

A bone mount 240 may be disposed on the patient 216. In some embodiments of the present disclosure, the bone mount 240 may be or comprise, for example, a clamp attached to a spinous process of a vertebra, a threaded rod capable of screwing into the patient table 204, or the like. In some embodiments of the present disclosure, the bone mount 240 may be connected to the patient 216 such that any movement of the patient 216 and/or the anatomical elements 220A-220D may result in a signal being sent to the robotic arm 116 to which the bone mount interface 250 and the bone mount 240 are connected. A movement of an anatomical element 220D in a first distance in a first direction may, for example, cause a signal to be sent to the robotic arm 116 indicating that the patient and/or anatomical element 220D moved the first distance in the first direction.

Attaching the bone mount interface 250 with the bone mount 240 creates a bone mount end effector 290. Thus, the bone mount end effector 290 includes the bone mount interface 250 and the bone mount 240 securely attached together. The bone mount interface 250 is configured to provide a stable and secure attachment while still allowing for a wide range of motion and flexibility. According to embodiments of the present disclosure, the bone mount end effector 290 can include various locking mechanisms between the bone mount interface 250 and the bone mount 240 for easy connection and reconnection. The locking mechanism may include a clamp, a screw, a ball and socket, a kinematic interface, etc. to ensure that the bone mount 240 remains in place during the surgical procedure. According to embodiments of the present disclosure, the kinematic interface may include the combination of a kinematic mount and a kinematic mount contact. The kinematic mount may correspond to one or more kinematic mounts including, but in no way limited to, chamfered slots, conical recesses, countersunk holes, counterbores, parallel dowel pins disposed in a slot offset a distance from one another, hardened slots, and/or combinations thereof. The kinematic mount contact may correspond to one or more contacts including, but in no way limited to, spherical balls, tooling balls with posts, dowel pins, and/or other protrusions. Moreover, the locking mechanism can be a manual locking mechanism, or an electronic locking mechanism and the locking mechanism connects easily and without forcing movement. According to an alternative embodiment of the present disclosure, a force or torque sensor is included that is configured to detect any skiving relative to the patient anatomy.

By using the robotic arm 116 with the bone mount end effector 290, surgeons can perform delicate and complex procedures with a greater degree of accuracy, reducing the risk of complications and improving patient outcomes. The bone mount interface 250 may be compatible with a variety of different bone mount 240 designs in order to accommodate different surgical procedures and patient needs. A robotic arm 116 with the bone mount end effector 290 has numerous advantages over traditional surgical methods. A robotic arm 116 with the bone mount end effector 290 can provide greater precision and accuracy, reduce the risk of human error and allow for less invasive procedures that result in faster recovery times and fewer complications.

According to embodiments of the present disclosure, mounting the robotic arm 116 with reference to a patient's anatomy allows the robotic systems to have a better accuracy than robotic systems that are not patient mounted. When enabling the bone mount 240 to a patient anatomy 220 at the end effector 224 of the robotic arm 116, a higher degree of accuracy can be achieved. This higher degree of accuracy is achieved by the relative geometry of the patient anatomy 220 (e.g., vertebra) and the end effector 224 being locked in place.

Moreover, the bone mount interface 250 enables a simple connection with the patient 216 since the bone mount interface 250 can be used with a variety of bone mounts 240. Furthermore, the bone mount interface 250, when attached to standard robotic arms 116, adds an additional DOF to the standard robotic arms 116 with little to no reconfiguration of the standard robotic arms 116. According to one embodiment of the present disclosure, the additional DOF is an added rotational DOF at the end effector 224 after an arm guide of the robotic arm. In this case the arm guide remains stationary. According to an alternative embodiment of the present disclosure, the additional DOF is an added cartesian DOF at the end effector 224 after the arm guide. In this case also, the arm guide remains stationary.

In one embodiment of the present disclosure, the bone mount 240 includes orientation capabilities. Therefore, a registration process can register the location of the bone mount 240. In the registration process, the orientation of the vertebra 220 to the bone mount is determined. This determination is made in several ways. After the registration process, the location of the bone mount interface 250 relative to the bone mount 240 is measured. Accordingly, a surgical procedure on one or two vertebrae 220 above or below where the bone mount 240 is located may be performed.

The registration of the bone mount 240 is not the key feature of the present disclosure. For example, a reference frame to the bone mount 240 is first registered. Afterwards, the navigation system 118 such as an optical navigation system using the optical sensor 228, determines the location of the robotic arm 116. According to embodiments of the present disclosure, the incorporation of the bone mount interface 250 enhances the features of the optically navigated robotic arm 116. Moreover, since the bone mount interface 250 is used with the navigation system 118 to determine location, various types of materials such as metal, plastic, etc. can be used for the bone mount 240.

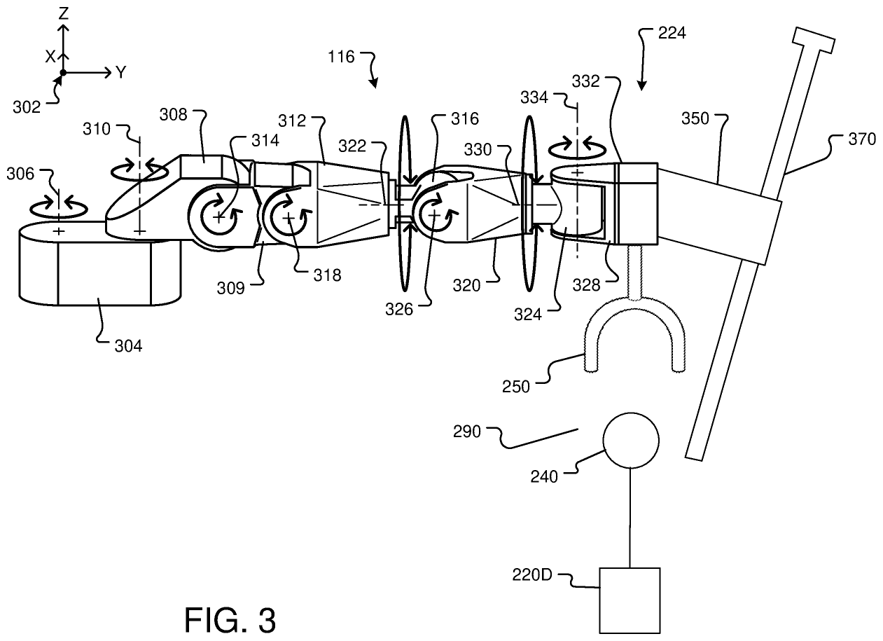

FIG. 3 is a perspective diagram of the robotic arm 116 with a bone mount end effector 290 according to at least one embodiment of the present disclosure. More specifically, FIG. 3 shows the robotic arm 116 of the robot 114 connected to the end effector 224 including the bone mount interface 250 attached thereto. The bone mount interface 250 is positioned within tool block 332. Features of the robot 114 and/or robotic arm 116 may be described in conjunction with a coordinate system 302. The coordinate system 302, as shown in FIG. 3 includes three-dimensions comprising an X-axis, a Y-axis, and a Z-axis. Additionally, or alternatively, the coordinate system 302 may be used to define planes (e.g., the XY-plane, the XZ-plane, and the YZ-plane) of the robot 114 and/or robotic arm 116. These planes may be disposed orthogonal, or at 90 degrees, to one another. While the origin of the coordinate system 302 may be placed at any point on or near the components of the robot 114, for the purposes of description, the axes of the coordinate system 302 are always disposed along the same directions from figure to figure, whether the coordinate system 302 is shown or not. In some examples, reference may be made to dimensions, angles, directions, relative positions, and/or movements associated with one or more components of the robot 114 and/or robotic arm 116 with respect to the coordinate system 302. For example, the width of the robotic arm 116 (e.g., running from the side shown in the foreground to the side in the background, into the page) may be defined as a dimension along the X-axis of the coordinate system 302, the height of the robotic arm 116 may be defined as a dimension along the Z-axis of the coordinate system 302, and the length of the robotic arm 116 (e.g., running from a proximal end at the first link 304 to a distal end at the seventh link 324, etc.) may be defined as a dimension along the Y-axis of the coordinate system 302. Additionally, or alternatively, the height of the system 100 may be defined as a dimension along the Z-axis of the coordinate system 302, a reach of the robotic arm 116 may be defined as a dimension along the Y-axis of the coordinate system 302, and a working area of the robotic arm 116 may be defined in the XY-plane with reference to the corresponding axes of the coordinate system 302.

The robotic arm 116 may be comprised of a number of links 304, 308, 309, 312, 316, 320, 324 that interconnect with one another at respective axes of rotation 306, 310, 314, 318, 322, 326, 330, 334, or joints. There may be more or fewer links 304, 308, 309, 312, 316, 320, 324 and/or axes of rotation 306, 310, 314, 318, 322, 326, 330, 334 than are shown in FIG. 3. In any event, the robotic arm 116 may have a first link 304 disposed at a proximal end of the robotic arm 116 and an end mount flange 328 disposed furthest from the proximal end at a distal end of the robotic arm 116. The first link 304 may correspond to a base of the robotic arm 116. In some examples, the first link 304 may rotate about first rotation axis 306. A second link 308 may be connected to the first link 304 at a second rotation axis 310, or joint. The second link 308 may rotate about the second rotation axis 310. In one example, the first rotation axis 306 and the second rotation axis 310 may be arranged parallel to one another. For instance, the first rotation axis 306 and the second rotation axis 310 are shown extending along the Z-axis in a direction perpendicular to the XY-plane.

The robotic arm 116 may comprise a third link 309 that is rotationally interconnected to the second link 308 via the third rotation axis 314, or joint. The third rotation axis 314 is shown extending along the X-axis, or perpendicular to the first rotation axis 306 and second rotation axis 310. In this position, when the third link 309 is caused to move (e.g., rotate relative to the second link 308), the third link 309 (and the components of the robotic arm 116 extending from the third link 309) may be caused to move into or out of the XY-plane. The fourth link 312 is shown rotationally interconnected to the third link 309 via the fourth rotation axis 318, or joint. The fourth rotation axis 318 is arranged parallel to the third rotation axis 314. The fourth rotation axis 318 extends along the X-axis allowing rotation of the fourth link 312 into and out of the XY-plane.

In some examples, the robotic arm 116 may comprise one or more wrists 316, 324. The fifth link 316, or wrist, is shown rotationally interconnected to the fourth link 312 via a fifth rotation axis 322, or wrist joint. The fifth rotation axis 322 is shown extending along the Y-axis, which is perpendicular to the X-axis and the Z-axis. During operation of the robot 114, causing the fifth link 316 to rotate about the fifth rotation axis 322 may cause the components of the robotic arm 116 distal the joint at the fifth rotation axis 322 (e.g., the fifth link 316, the sixth link 320, the seventh link 324, the end mount flange 328, and the end effector 224, etc.) to rotate about the Y-axis.

The sixth link 320 is rotationally interconnected to the fifth link 316 via the sixth rotation axis 326. The sixth rotation axis 326 extends along the X-axis and provides for rotation of the sixth link 320 relative to the fifth link 316 (e.g., into and out of the XY-plane in the position shown).

The seventh link 324, or wrist, is shown rotationally interconnected to the sixth link 320 via a seventh rotation axis 330, or wrist joint. The seventh rotation axis 330 is shown extending along the Y-axis (e.g., perpendicular to the X-axis and the Z-axis). During operation of the robot 114, causing the seventh link 324 to rotate about the seventh rotation axis 330 may cause the components of the robotic arm 116 distal the joint at the seventh rotation axis 330 (e.g., the end mount flange 328, and the end effector 224, etc.) to rotate about the Y-axis.

Located at the distal end of the robotic arm 116, an end mount flange 328 may be rotationally interconnected to the end mount flange 328 via an eighth, or mount flange rotation, axis 334. As shown in FIG. 3, the seventh link 324 is positioned rotationally about the seventh rotation axis 330 such that the end mount flange 328 is oriented where the mount flange rotation axis 334 is extending along the Z-axis for one type of robotic arm 116 with one type of movement kinematics. According to an alternative embodiment of the present disclosure, the seventh link 324 is positioned rotationally about the seventh rotation axis 330 such that the end mount flange 328 is oriented where the mount flange rotation axis 334 is extending along the X-axis for another type of robotic arm 116 having another type of movement kinematics.

Robotic arm 116 may also include an arm guide 350 attached to the tool block 332 for example. The arm guide 350 may include a surgical tool 370. While shown as a single surgical tool 370 in FIG. 3, the surgical tool 370 may correspond to different surgical tools 370 used between operations in a surgical application. Although arm guide 350 as illustrated as being attached to the robotic arm 116 at one end of the tool block 332, arm guide 350, however, may be attached at any location on the robotic arm 116 so that the surgical tool 370 can be used to perform surgical applications. According to one embodiment of the present disclosure, the arm guide 350 may be provided in close proximity to and on the side of the end effector 224 of the robotic arm 116 at one or more locations.

Moreover, the bone mount interface 250, when attached to standard robotic arms 116 at or near the end effector 224, adds an additional DOF to the standard robotic arms 116 with little to no reconfiguration of the standard robotic arms 116. According to one embodiment of the present disclosure, the additional DOF is an added rotational DOF at the end effector 224 after the arm guide 350 of the robotic arm 116. In this case the arm guide 350 remains stationary. According to an alternative embodiment of the present disclosure, the additional DOF is an added cartesian DOF at the end effector 224 after the arm guide 350. In this case also, the arm guide 350 also remains stationary. Moreover, and according to embodiments of the present disclosure, although the bone mount 240 physically connects the patient to the robot 114 (e.g., the robotic system) operating procedures are still guided by the robotic system. Thus, computing device 102 determines the position of the robotic arm 116 in order to connect the bone mount 240 (the position of the bone mount 240 is determined by the navigation system 118, the imaging device(s) 112 or other systems) and in order to direct or point the robot 114 in the correct trajectory to allow an operating procedure to take place (e.g., place a pedicle screw). This is not an easy task mathematically or physically. Thus, the bone mount end effector according to embodiments of the present disclosure is very forgiving and flexible and can allow for many configurations such as a ball and socket configuration, for example.

According to further embodiments of the present disclosure, for a specific bone mount 240, the robotic arm 116 can attach to the specific bone mount 240 in several different directions. Being able to attach to a specific bone mount 240 in several different directions allows for the physical connection (e.g., the robot 114 may be limited in motion and can connect to the bone mount 240 only in specific directions) and allows for multiple operating procedures (e.g., several screw placements) with the same bone mount interface 250 detaching and reattaching the same or different bone mounts 240. The bone mount end effector according to embodiments of the present disclosure allows for less risk of losing registration and for greater safety for the patient. Moreover, the bone mount end effector according to embodiments of the present disclosure allows for less risk to loosen connection of the bone mount 240.

According to further embodiments of the present disclosure, the bone mount 240 can be tracked via the navigation system 118, the imaging device(s) 112 or other systems using a tracking marker for example.

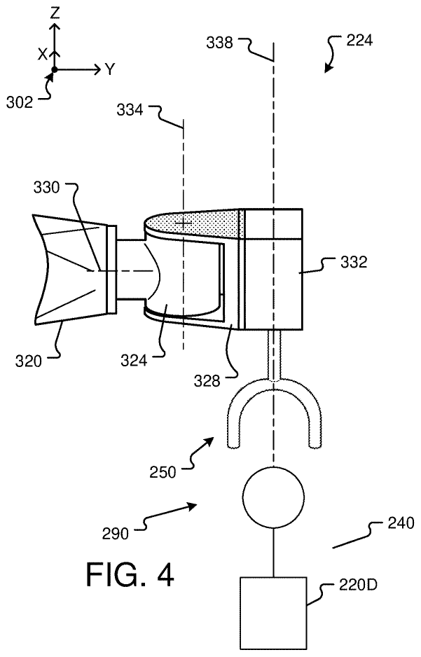

FIG. 4 is a detail perspective view of a portion of the robotic arm 116 with the bone mount end effector 290 according to at least one embodiment of the present disclosure. As illustrated in FIG. 4, the end effector 224 may include the tool block 332 having a receptacle disposed therein (not shown) for receiving the bone mount interface 250. The receptacle may define a tool axis 338 of the tool block 332. In one example, an axis of the receptacle may coincide with the tool axis 338. When attached to the end mount flange 328, the tool block 332 disposes the tool axis 338 parallel to the mount flange rotation axis 334. In an alternative embodiment of the present disclosure (not shown), the robotic arm 116 may be configured such that when the tool block 332 is attached to the end mount flange 328, the tool block 332 disposes the tool axis 338 perpendicular to the mount flange rotation axis 334.

According to embodiments of the present disclosure, the bone mount interface 250, when attached to the robotic arm 116 when the tool block 332 disposes the tool axis 338 parallel to the mount flange rotation axis 334 or when the tool block 223 disposes the tool axis 338 perpendicular to the mount flange rotation axis 334, adds the additional DOF to these different types of robotic arms 116 with little to no reconfiguration of the standard robotic arms 116.

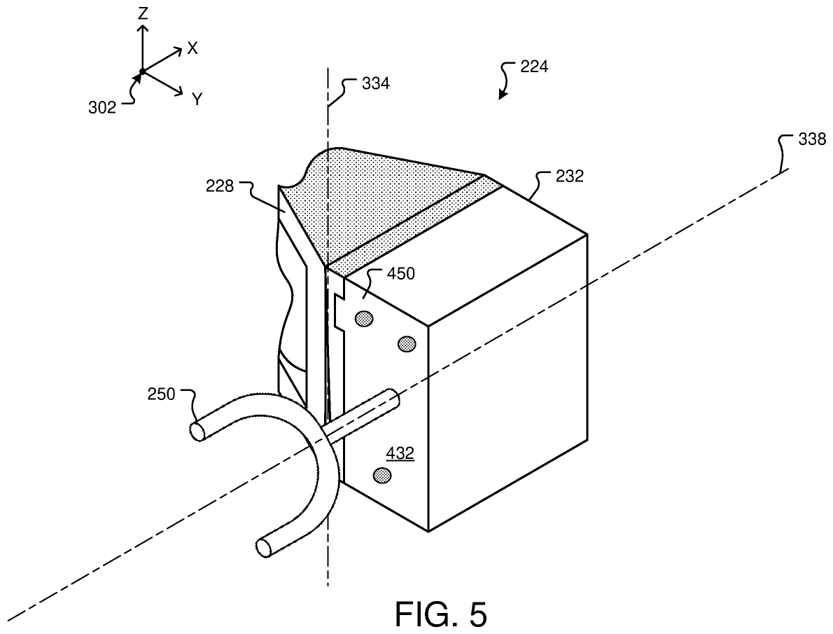

FIG. 5 is a detail perspective view of a portion of the robotic arm 116 with the end effector 224 including the bone mount interface 250 according to at least one embodiment of the present disclosure. As illustrated in FIG. 5, the portion of the robotic arm 116 is configured such that when the tool block 332 is attached to the end mount flange 328, the tool block 332 disposes the tool axis 338 perpendicular to the mount flange rotation axis 334. On one face 432 of the tool block 332 there is provided the bone mount interface 250 along with one or more sensors 450. The one or more sensors 450 monitor the location of the bone mount 240 with respect to the bone mount interface 250. Thus, the one or more sensors 450 provide feedback to the robotic arm 116, allowing the robotic arm 116 to adjust its position and orientation based on movement of the patient, the breathing of the patient, the movement of the patient bone, etc.

Moreover, the one or more sensors 450 along with a feedback mechanism are provided such that the bone mount interface 250 is released from the bone mount 240 if a threshold level of force or distance is received by the processor 104 of the robot 114. Also, the feedback mechanism may be provided such that an alarm is caused to be generated by the processor 104 of the robotic system 114 if a threshold level of force exerted by the surgeon or the robotic arm 116 or a threshold distance is displaced by the bone mount 240 is received by the processor 104.

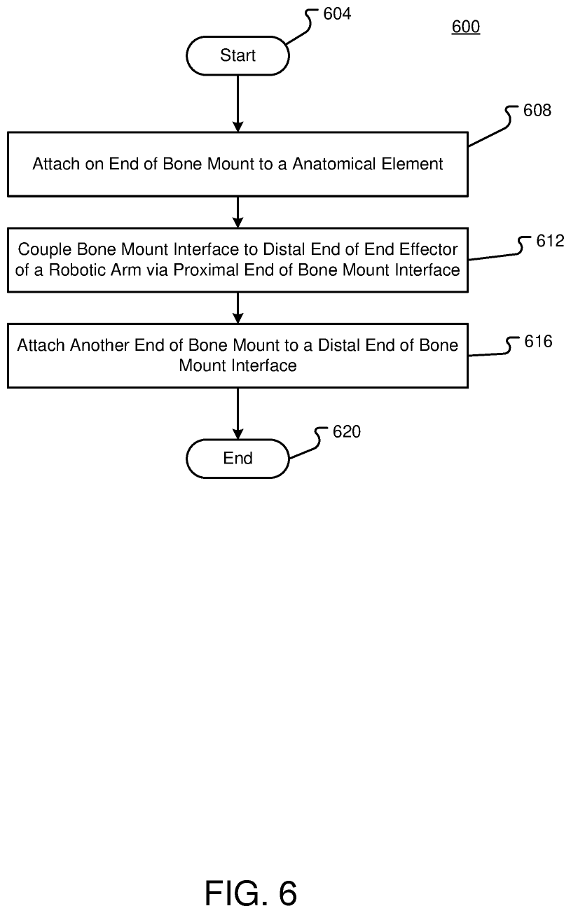

FIG. 6 depicts a method 600 that may be used, for example, in operating the robot 114 and/or one or more portions of the robotic arm 116 with the bone mount end effector 290. The method 600 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 600. The at least one processor may perform the method 600 by executing elements stored in a memory such as the memory 106. The elements stored in the memory and executed by the processor may cause the processor to execute one or more steps of a function as shown in method 600. One or more portions of a method 600 may be performed by the processor executing any of the contents of memory, such as transformation 124 and/or registration 128 instructions.

FIG. 6 is a flowchart of a method 600 for operating the robotic arm with the bone mount end effector 290 according to at least one embodiment of the present disclosure. For example, the method 600 provides for a rigid connection between the end effector 224 of the robot 114 and a bone mount 240 placed on a patient 216 and allows for the most accurate registration of the patient 216 to the robotic arm 116 according to embodiments of the present disclosure. While a general order for the steps of the method 600 is shown in FIG. 6, the method 600 can include more or fewer steps or can arrange the order of the steps differently than those shown in FIG. 6. Generally, the method 600 starts with a START operation at step 604 and ends with an END operation at step 620. The method 600 can be executed as a set of computer-executable instructions executed by an assembly machine (e.g., robotic assembly system, automation assembly system, computer aided drafting (CAD) machine, etc.) and encoded or stored on a computer readable medium. Hereinafter, the method 600 shall be explained with reference to the components, devices, assemblies, environments, etc. described in conjunction with FIGS. 1-5.

The method 600 may begin with the START operation at step 604 and proceeds to step 608 where one end of a bone mount 240 is attached to an anatomical element 220. According to embodiments of the present disclosure, the anatomical element 220 may be vertebra. After one end of a bone mount 240 is attached to the anatomical element 220 at step 608, method 600 proceeds to step 612, where a bone mount interface 250 is coupled to a distal end of the end effector 224 of the robotic arm 116 via a proximal end of the bone mount interface 250. After the bone mount interface 250 is coupled to a distal end of an end effector 224 of the robotic arm 116 via a proximal end of the bone mount interface 250 at step 612, method 600 proceeds to step 616, where the other end of the bone mount 240 is attached to a distal end of the bone mount interface 250. According to embodiments of the present disclosure, with this arrangement of the bone mount 240 and the bone mount interface 250 coupled together, a bone mount end effector 290 is configured. The bone mount interface 250 is configured to add an additional DOF to the end effector 224.

After the other end of the bone mount 240 is attached to a distal end of the bone mount interface 250 at step 616, method 600 may end with the END operation at step 620. According to further embodiments of the present disclosure, one or more sensors 450 along with a feedback mechanism are provided on the end effector 224 such that the bone mount interface 250 is released from the bone mount 240 if a threshold level of force or distance is received by the processor 104 of the robot 114. Also, the feedback mechanism may be provided such that an alarm is caused to be generated by the processor 104 of the robot 114 if a threshold level of force exerted by the surgeon or the robotic arm 116 or a threshold distance is displaced by the bone mount 240 is received by the processor 104.

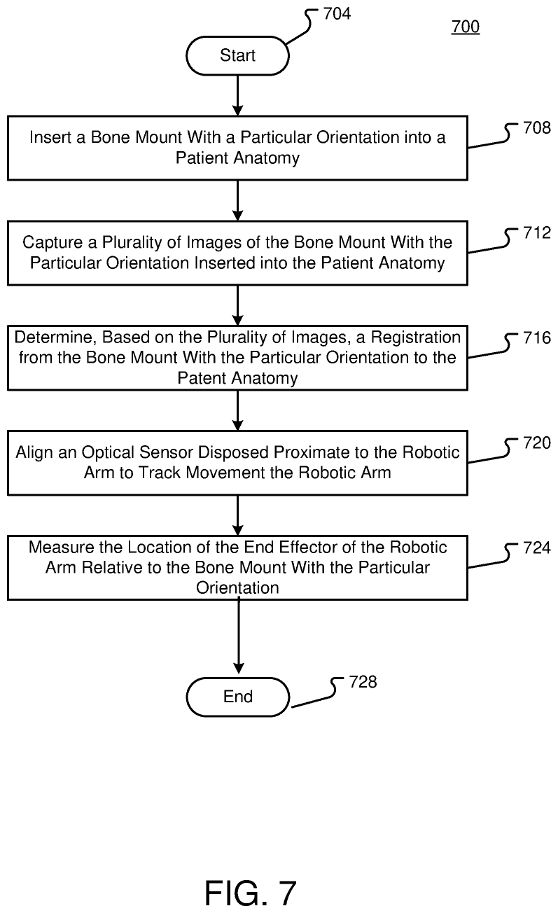

FIG. 7 is a flowchart of a method 700 for measuring a location of an end effector 224 of a robotic arm 166 relative to a bone mount 240 and navigating the robotic arm relative to a patient anatomy using the bone mount 240 according to at least one embodiment of the present disclosure. FIG. 7 depicts a method 700 that may be used, for example, in operating the robot 114 and/or one or more portions of the robotic arm 116 having a bone mount end effector 290.

The method 700 (and/or one or more steps thereof) may be carried out or otherwise performed, for example, by at least one processor. The at least one processor may be the same as or similar to the processor(s) 104 of the computing device 102 described above. The at least one processor 104 may be part of a robot (such as a robot 114) or part of a navigation system (such as a navigation system 118). A processor other than any processor described herein may also be used to execute the method 700. The at least one processor may perform the method 700 by executing elements stored in a memory such as the memory 106. The elements stored in the memory and executed by the processor may cause the processor to execute one or more steps of a function as shown in method 700. One or more portions of a method 700 may be performed by the processor executing any of the contents of memory, such as image processing 120, segmentation 122, transformation 124 and/or registration 128 instructions.

While a general order for the steps of the method 700 is shown in FIG. 7, the method 700 can include more or fewer steps or can arrange the order of the steps differently than those shown in FIG. 7. Generally, the method 700 starts with a START operation at step 704 and ends with an END operation at step 728. The method 700 can be executed as a set of computer-executable instructions executed by an assembly machine (e.g., robotic assembly system, automation assembly system, computer aided drafting (CAD) machine, etc.) and encoded or stored on a computer readable medium. Hereinafter, the method 700 shall be explained with reference to the components, devices, assemblies, environments, etc. described in conjunction with FIGS. 1-5.

The method 700 may begin with the START operation at step 704 and proceed to step 708 where a bone mount 240 with a particular orientation is inserted into a patient anatomy 220. After the bone mount 240 with the particular orientation has been inserted into a patient anatomy 220 at step 708, method 700 proceeds to step 712, where a plurality of images of the bone mount 240 with the particular orientation inserted into the patient anatomy 220 are captured. In some embodiments of the present disclosure, the bone mount 240 may include a marker, such as one or more fluoroscopic markers. The plurality of images may be or comprise a plurality of fluoroscopic images or fluoroscopic image data captured by a fluoroscopic imaging device (e.g., an X-ray source and an X-ray detector). In some embodiments of the present disclosure, the images may be captured using an O-arm or other imaging device 112. The fluoroscopic imaging device may be positioned at a predetermined location when each image is captured, and each captured image of the plurality of images may depict the one or more fluoroscopic markers in a different pose (e.g., a different position and/or orientation).

According to an alternative embodiment of the present disclosure, the bone mount 240 may not include markers. In this case, the plurality of images of the bone mount 240 which may be made of metal or plastic, are captured, and identified from the images. In some embodiments of the present disclosure, the plurality of images may be captured preoperatively (e.g., before the surgery or surgical procedure begins).

After the plurality of images of the bone mount 240 with the particular orientation inserted into the patient anatomy 220 are captured at step 712, method 700 proceeds to step 716, where a registration from the bone mount with the particular orientation to the patient anatomy 220 is determined based on the plurality of images. The registration may be or comprise a map from the coordinates associated with one or more of the fluoroscopic markers in a first coordinate system to a second coordinate system containing the coordinates of the patient element 220, or vice versa. In some embodiments of the present disclosure, the registration may transform both sets of coordinates into a third coordinate system, such as a coordinate system used by the robotic arm 116. In some embodiments of the present disclosure, the plurality of images may depict additional patient elements 220 (e.g., multiple vertebrae of the spine), and the registration may include mapping coordinates associated with each of the additional patient elements 220 into a common coordinate system.

In some embodiments of the present disclosure, the registration may be determined using image processing 120 and one or more registrations 128. The image processing 120 may be used to identify the one or more fluoroscopic markers and the patient element 220 in each image of the plurality of images. In some embodiments of the present disclosure, the image processing 120 may be or comprise one or more machine learning and/or artificial intelligence models that receive each image as an input and output coordinates associated with each identified fluoroscopic marker and patient element 220. The registration 128 may use the determined coordinates associated with each identified fluoroscopic marker and the patient element 220 to determine a pose of each fluoroscopic marker relative to the patient element 220 and may transform the coordinates associated with each fluoroscopic marker from a first coordinate system to a second coordinate system. For example, the registration 128 may take coordinates associated with each of the identified fluoroscopic markers and map the coordinates into a coordinate system associated with the patient element 220 (or vice versa). Additionally, or alternatively, the registration 128 may map the fluoroscopic marker coordinates and the patient element 220 coordinates into a third coordinate system (e.g., a robotic space coordinate system) shared by other surgical tools or components in a surgical environment.

After the registration from the bone mount 240 with the particular orientation to the patient anatomy 220, based on the plurality of images is determined at step 716, method 700 proceeds to step 720 where an optical sensor 228 disposed proximate to the robotic arm 116 is used to track movement of the robotic arm 116. The optical sensor 228 may be pointed at the tracking marker 232 provided on the end effector 224 of the robotic arm 116 such that the tracking marker 232 can be identified and a pose of the tracking marker 232 can be determined. For example, the optical sensor 228 may be or comprise a laser tracker that emits a laser that is captured by the tracking marker 232 (e.g., an optical tracking marker).

Alternatively, the optical sensor 228 may be or comprise a 3D camera, and the tracking marker 232 may be or comprise a 3D tracking target. In some embodiments of the present disclosure, the optical sensor 228 may automatically identify the tracking marker 232 (e.g., the processor 104 may cause the optical sensor 228 to search the surrounding area until the tracking marker 232 is identified), or the optical sensor 228 may alternatively be aligned manually (e.g., by a physician, by a member of the surgical staff, etc.). In some embodiments of the present disclosure, the alignment of the optical sensor 228 with the tracking marker 232 may occur after preoperative images (e.g., the plurality of images) are captured.

After the optical sensor 228 disposed proximate to the robotic arm 116 is used to track movement of the robotic arm 116 at step 720, method 700 proceeds to step 724, where the location of the end effector 224 of the robotic arm 116 relative to the bone mount 240 with the particular configuration is measured. After the location of the end effector 224 of the robotic arm 116 relative to the bone mount 240 with the particular configuration is measured at step 724, method 700 ends with the END operation at step 728.

As noted above, the present disclosure encompasses methods with fewer than all of the steps identified in FIGS. 6 and 7 (and the corresponding description of the methods 600 and 700), as well as methods that include additional steps beyond those identified in FIGS. 6 and 7 (and the corresponding description of the methods 600 and 700). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example 1. A system, comprising:
a robotic arm;
an end effector having a proximal end and a distal end,
wherein the proximal end of the end effector is connected to the robotic arm;
a bone mount attachable to an anatomical element at one end of the bone mount; and
a bone mount interface coupled to the distal end of the end effector via a proximal end of the bone mount interface and attached to another end of the bone mount via a distal end of the bone mount interface,
wherein the bone mount interface is configured to add a degree of freedom to the end effector.

Example 2. The system of example 1, wherein the anatomical element includes one or more vertebrae.

Example 3. The system of example 1, further comprising one or more sensors (450) disposed at the distal end of the end effector.

Example 4. The system of example 3, wherein the one or more sensors include a force/torque sensor disposed at the distal end of the end effector configured to output a force signal in accordance with a force exerted on the bone mount.

Example 5. The system of example 4, further comprising control circuitry configured to receive the force signal and output a control signal based on the force exerted on the bone mount.

Example 6. The system of example 5, wherein the control signal includes activating an alarm when the force signal exceeds a predetermined threshold.

Example 7. The system of example 5, wherein the control signal includes releasing the bone mount from the bone mount interface when the force signal exceeds a predetermined threshold.

Example 8. The system of example 3, wherein the one or more sensors provide feedback to the robotic arm, allowing the robotic arm to adjust its position and orientation based on movement of the anatomical element.

Example 9. The system of example 1, wherein the bone mount includes a locking mechanism configured to be selectively and repeatedly coupled to and uncoupled from the bone mount interface.

Example 10. The system of example 1, wherein the bone mount includes a ball and socket mechanism.

Example 11. The system of example 1, wherein the degree of freedom is a rotational degree of freedom.

Example 12. The system of example 1, wherein the degree of freedom is a translational degree of freedom.

Example 13. The system of example 1, wherein the bone mount interface is mechanically coupled to the distal end of the end effector to provide a rigid connection between the end effector and the bone mount.

Example 14. The system of example 1, wherein the bone mount includes a kinetic interface or a clamping interface.

Example 15. The system of example 1, further comprising an arm guide provided between a proximal end and a distal end of the robotic arm and adjacent to the end effector, wherein the arm guide is configured to accommodate a surgical tool.

Example 16. A system, comprising:
one or more processors;
at least one robotic arm; and
a memory storing data for processing by the one or more processors that, when processed by the one or more processors, causes the one or more processors to:
   determine, based on first sensor data, a first force exerted by a bone mount interface onto a bone mount attached to an anatomical element,
   wherein the bone mount interface is couple to an end effector of the at least one robotic arm;
   determine, based on second sensor data, a second force different from the first force, exerted by the bone mount interface onto the bone mount; and
   output a control signal based on the second force exerted on the bone mount.

Example 17. The system of example 16, wherein the control signal includes activating an alarm when the second force exceeds a predetermined threshold.

Example 18. The system of example 16, wherein the control signal includes releasing the bone mount from the bone mount interface when the second force exceeds a predetermined threshold.

Example 19. A method, comprising:
attaching one end of a bone mount to an anatomical element;
coupling a bone mount interface to a distal end of an end effector of a robotic arm via a proximal end of the bone mount interface; and
attaching another end of the bone mount to a distal end of the bone mount interface,
wherein the bone mount interface is configured to add a degree of freedom to the end effector.

Example 20. The method of example 19, further providing one or more sensors at the distal end of the end effector.

1-15. (canceled)

16. A system, comprising:
a robotic arm;
an end effector having a proximal end and a distal end,
wherein the proximal end of the end effector is connected to the robotic arm;
a bone mount attachable to an anatomical element at one end of the bone mount; and
a bone mount interface coupled to the distal end of the end effector via a proximal end of the bone mount interface and attached to another end of the bone mount via a distal end of the bone mount interface,
wherein the bone mount interface is configured to add a degree of freedom to the end effector.

17. The system of claim 16, wherein the anatomical element includes one or more vertebrae.

18. The system of claim 16, further comprising one or more sensors (450) disposed at the distal end of the end effector.

19. The system of claim 18, wherein the one or more sensors include a force/torque sensor disposed at the distal end of the end effector configured to output a force signal in accordance with a force exerted on the bone mount.

20. The system of claim 19, further comprising control circuitry configured to receive the force signal and output a control signal based on the force exerted on the bone mount.

21. The system of claim 20, wherein the control signal includes activating an alarm when the force signal exceeds a predetermined threshold.

22. The system of claim 20, wherein the control signal includes releasing the bone mount from the bone mount interface when the force signal exceeds a predetermined threshold.

23. The system of claim 18, wherein the one or more sensors provide feedback to the robotic arm, allowing the robotic arm to adjust its position and orientation based on movement of the anatomical element.

24. The system of claim 16, wherein the bone mount includes a locking mechanism configured to be selectively and repeatedly coupled to and uncoupled from the bone mount interface.

25. The system of claim 16, wherein the bone mount includes a ball and socket mechanism.

26. The system of claim 16, wherein the degree of freedom is a rotational degree of freedom.

27. The system of claim 16, wherein the degree of freedom is a translational degree of freedom.

28. The system of claim 16, wherein the bone mount interface is mechanically coupled to the distal end of the end effector to provide a rigid connection between the end effector and the bone mount.

29. The system of claim 16, wherein the bone mount includes a kinetic interface or a clamping interface.

30. The system of claim 16, further comprising an arm guide provided between a proximal end and a distal end of the robotic arm and adjacent to the end effector, wherein the arm guide is configured to accommodate a surgical tool.

31. A system, comprising:
one or more processors;
at least one robotic arm; and
a memory storing data for processing by the one or more processors that, when processed by the one or more processors, causes the one or more processors to:
determine, based on first sensor data, a first force exerted by a bone mount interface onto a bone mount attached to an anatomical element,
wherein the bone mount interface is couple to an end effector of the at least one robotic arm;
determine, based on second sensor data, a second force different from the first force, exerted by the bone mount interface onto the bone mount; and
output a control signal based on the second force exerted on the bone mount.

32. The system of claim 31, wherein the control signal includes activating an alarm when the second force exceeds a predetermined threshold.

33. The system of claim 31, wherein the control signal includes releasing the bone mount from the bone mount interface when the second force exceeds a predetermined threshold.

34. A method, comprising:
attaching one end of a bone mount to an anatomical element;
coupling a bone mount interface to a distal end of an end effector of a robotic arm via a proximal end of the bone mount interface; and
attaching another end of the bone mount to a distal end of the bone mount interface,
wherein the bone mount interface is configured to add a degree of freedom to the end effector.

35. The method of claim 34, further providing one or more sensors at the distal end of the end effector.
